(19) United States
(12) Patent Application Publication
(10) Pub. No.: US 2016/0151300 A1
(43) Pub. Date: Jun. 2, 2016

(54) Method of Producing and Introducing a Far Infrared Device By Way of Adhesive Tape

(71) Applicant: jeremy madvin, Westlake Village, CA (US)

(72) Inventor: jeremy madvin, Westlake Village, CA (US)

(21) Appl. No.: 14/555,860

(22) Filed: Nov. 28, 2014

(51) Int. Cl. A61K 9/70; A61K 33/32; A61K 33/26; A61K 33/10; A61K 33/34; A61K 33/22; A61K 33/00; A61K 33/08; A61K 41/00; A61K 33/30

(52) U.S. Cl. CPC A61K 9/7038; A61K 41/00; A61K 33/32; A61K 33/26; A61K 33/30; A61K 33/34; A61K 33/22; A61K 33/00; A61K 33/08; A61K 33/10

(57) ABSTRACT

The present invention relates to a safe, dermatologically safe device in the form of tape containing far-infrared generating particles and a method for producing such a product. The method for producing the far-infrared tape consists of mixing fine particles of far-infrared emitting substances into an adhesive or the tape backing material prior to or following further processing such as, but not limited to forming, molding, filling, pressing, dipping or extruding the material. The substances generating far-infrared radiation are to consist of safe substances such as, but not limited to, Magnesium, Manganese, Germanium, Iron, Zinc, Copper, Tourmaline, Silicone Dioxide, Aluminum Dioxide, Germanium, Iron Oxide, Calcium Carbonate or mixture thereof. Other ingredients can be included to further add to the health benefits of this invention.

### BACKGROUND

Far Infrared is commonly used for improving the health of a person or other animals, however, the approach has included sources external to the body, for example, clothing, support wraps, bedding, patches, heaters, and/or (b) been suggested that the use of nano particles be used for this type of application.

This invention allows a far infrared emitting device to be produced and introduced externally, without the required use of nano particles, for the purposes of offering the benefits available through far infrared, including potentially increasing blood flow deeper in the body than non far infrared external sources in effort to service and biologically effect aspects of the circulatory system, organs and other components of the animal's internal systems.

### BRIEF DESCRIPTION OF THE DRAWINGS

FIG. 1 is a perspective view of the Tape with its adhesive layer including a far infrared material.

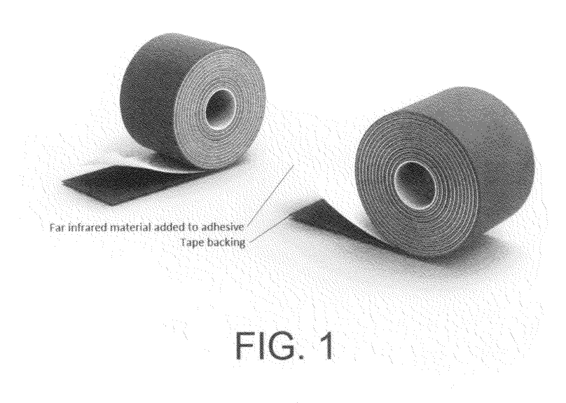

FIG. 2 is a perspective view of the Tape with both its adhesive layer including a far infrared material and an outer coating or surface treatment on the fabric including far infrared material.

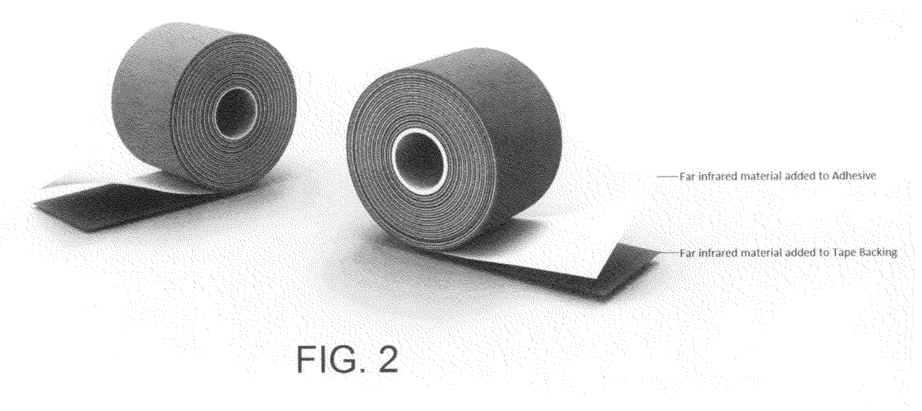

FIG. 3 is a perspective view of the Tape showing an outer coating or surface treatment on the fabric having a fine dispersion of far-infrared generating substance as the key ingredient of said coating.

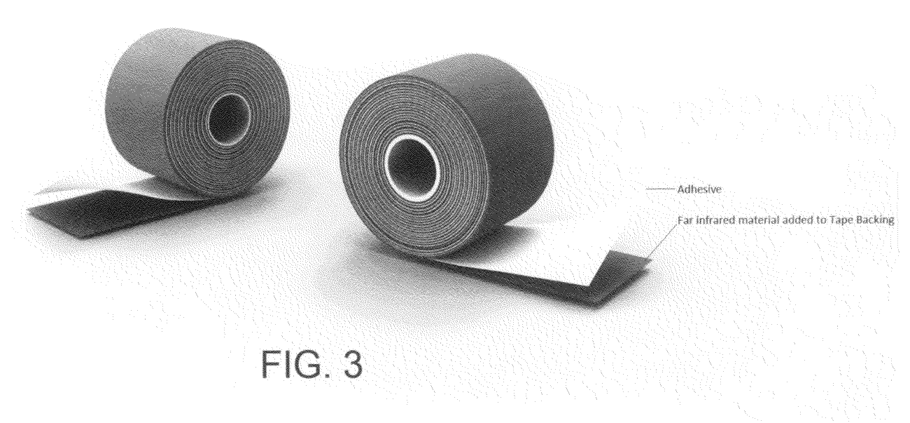

### BRIEF SUMMARY OF THE INVENTION

While conventional devices in the form of medical tape, including kinetic, tape are used for a multitude of purposes, no device in the form of tape incorporating a group of substances which can be safely used to generate far-infrared and blended into the adhesive or tape backing have been marketed. The incorporation of the far-infrared generating substances into the adhesive or backing provides for various health promoting benefits including improved circulation, aiding in stress relief, accelerating recovery from fatigue and injuries and increasing mental alertness and detoxification. In addition, other ingredients may be added which offer additional health benefits, such as increased promotion of detoxification, support of heart health and so on.

The object of the present invention is to provide a novel adhesive or backing used to produce a tape for application on or near an animals skin. This device can generate measurable far-infrared with emissivity greater than 0.7 but below 0.97 that serve to promote the health or general wellness of the user.

In addition, unlike a conventional adhesive patch, this invention would require the tape to be longer than eight inches and no wider than 4 inches. This enables the user to be able to create countless options for length and shape of that tape which is applied to or near body.

The present invention provides a method of producing a health promoting device in the form of adhesive tape. Said method is comprised of producing an infused binder or filled housing by blending or adding finely pulverized powder or liquid of a dermatologically safe substance or combination of safely consumable substances intended to generate far-infrared into the adhesive or backing prior to processing through standard techniques such as forming, molding, extruding, pressing, loading and other common practices of adhesive tape production.

Preferably, the adhesive or backing materials are made from but not limited to those which typically used in tape production, such as but not limited to, fabrics such as cotton or polyester, rubber, paper, cellulose acetate, hot melt glue or, acrylic depending on the final product. The finely ground far-infrared generating substances or liquid may be, but are not limited to a group consisting of Magnesium, Manganese, Iron, Zinc, Copper, Tourmaline, Silicone Dioxide, Aluminum Dioxide, Germanium, Iron Oxide, Calcium Carbonate or mixture thereof with an intended particle size of 0.5 microns or larger and occupying up to 95% of the total weight of final product.

### DETAILED DESCRIPTION AND BEST MODE OF IMPLEMENTATION

The present invention relates to a device in the form of a novel adhesive or backing, in adhesive tape form, suitable for direct contact with the skin. By adding or mixing a finely pulverized powder or liquid of far-infrared generating substances into and adhesive or backing material prior to or following the processing of such material, the finished product provides unique health-promoting benefits. Such substances would include, but are not limited to, Magnesium, Manganese, Germanium, Iron, Zinc, Copper, Tourmaline, Silicone Dioxide, Aluminum Dioxide, Iron Oxide, Calcium Carbonate or mixture thereof. Other ingredients may be added that offer additional health benefits, such as increased promotion of detoxification, provide analgesic effects or support of pursuits of bodily health.

Other inventions, mainly out of Japan and South Korea, have incorporated the use of far-infrared-producing materials by mixing these powders with synthetic fibers or resins to form a special material for creation of all types of products designed to be placed against or near the skin. Such products include clothing, bedding, support wraps, heating elements and similar items. It is not uncommon to add these powders to hard plastics to create discs for odor neutralization or EMF reduction, and it is not un-common to add the powders to coatings to spray on walls or other construction materials.

There is a significant body of literature indicating that far-infrared generating materials provide an overall invigorating and regenerative effect. Additionally, research in Japan and South Korea has shown far-infrared stone powder helps improve blood circulation, aid in stress relief, speed recovery from fatigue and injury, and increase mental alertness. The inventor believes similar wellness benefits would follow not only when the body is in close contact with the material, but also if this material could be adhered to the skin, for short or longer durations. In addition, a tape form allows the user to choose a specific length of shape of material to use. In addition, the tape can be used to provide significant support, comfort, compression and other benefits not found in other adhesive products, such as patches.

The inventor is not aware of any application where a powder or liquid consisting of far infrared substances is actually mixed into an adhesive or backing of the tape itself prior to the forming or further processing, so that the powder or liquid becomes integral to the adhesive or tape backing material with the intention of generating far-infrared once applied. The inventor believes there are many advantages to mixing or blending said powder or liquid directly into the base material prior to processing.

There is a body of substantiation that demonstrates that far infrared (a) is a nourishing energy found in nature, (b) is healthy for living tissue, and (c) penetrates the body 2-6 cm. In addition, it has been demonstrated that the strength of the far infrared is stronger at the source than distanced from the source. Furthermore, the farther the distance from the source, the weaker the far infrared emission.

These advantages of this invention include, but are not limited to, the ease of exposing any external part of the body to the source of the far-infrared emission in a safe and regulated fashion at a closer proximity to the source.

1. A device in the form of adhesive tape produced by forming, molding, extruding, pressing, loading, dipping or spraying, or other means and providing therapeutic benefits. The adhesive tape comprises: fabrics such as, but not limited to cotton or polyester, rubber, paper, cellulose acetate, hot melt glue or, acrylic depending on the final product. The finely ground far-infrared generating substances or liquid may be, but are not limited to a group consisting of Magnesium, Manganese, Iron, Zinc, Copper, Tourmaline, Silicone Dioxide, Aluminum Dioxide, Germanium, Iron Oxide, Calcium Carbonate or mixture thereof with an intended particle size of 0.5 microns or larger and occupying between up to 95% of the total weight of final product in size, that contains far-infrared generating substances; wherein said fine powder or liquid is dispersed and intermingled with said adhesive or backing material.

2. The adhesive of claim 1 wherein said fine powder further contains fine particles of other substances that may or may not contain far-infrared generating properties but are intended to add additional health benefits, such as, but not limited to, the promoting of detoxification, analgesic effects or pursuit of other health benefits.

3. The substances used to generate far-infrared must be measurable in terms of emission through spectrometry or another reliable means with emissivity greater than 0.7 but below 0.97 in order to serve to promote the health or general wellness of the user.

4. Tape to be longer than eight inches and no wider than 4 inches. This enables the user to be able to create countless options for length and shape of that tape which is applied to or near body.
